(19) United States
(12) Patent Application Publication
(10) Pub. No.: US 2013/0203121 A1
(43) Pub. Date: Aug. 8, 2013

(54) METHODS FOR THE SEMI-SYNTHETIC PRODUCTION OF HIGH PURITY "MINICIRCLE" DNA VECTORS FROM PLASMIDS

(71) Applicants: Bernd Rehberger, Laupheim (DE); Markus Heine, Braunschweig (DE); Claas Wodarczyk, Neu-Ulm (DE); Roland Wagner, Laupheim (DE)

(72) Inventors: Bernd Rehberger, Laupheim (DE); Markus Heine, Braunschweig (DE); Claas Wodarczyk, Neu-Ulm (DE); Roland Wagner, Laupheim (DE)

(73) Assignee: RENTSCHLER BIOTECHNOLOGIE GMBH, Laupheim (DE)

(21) Appl. No.: 13/877,586

(22) PCT Filed: Oct. 4, 2011
(86) PCT No.: PCT/EP2011/067280

§ 371 Date: Apr. 3, 2013

(30) Foreign Application Priority Data
Oct. 5, 2010 (EP) 10186568.1

(51) Int. Cl. C12P 19/34

(52) U.S. Cl. CPC C12P 19/34
USPC 435/91.4; 435/183

(57) ABSTRACT

The invention relates to methods and reagents for producing DNA vectors, in particular minicircle (MC) DNA vectors, in superhelical form. The invention further relates to highly pure preparations of circular DNA vectors, in particular MC DNA vectors.

The present invention relates to methods and reagents for producing DNA vectors, in particular minicircle (MC) DNA vectors, in superhelical form. The invention furthermore relates to high purity preparations of circular DNA vectors, in particular MC DNA vectors.

Previously used methods for producing circular minicircle DNA vectors are characterised by elevated complexity combined with low yields and considerable effort to achieve the necessary purity and quality (1, 3, 8, 11, 15).

A feature common to these methods is that they make use of recombinases in order to excise the MCs from the parental plasmids which have previously been multiplied in bacteria. On the one hand, using the recombinases ensures retention of the superhelical status of the MC which is required for subsequent use as a vector for transducing eukaryotic cells. On the other hand, this procedure is extremely inefficient, such that the yield of MCs, relative to the quantity of bacterial culture used, is very low. This is very largely because permanent expression of the recombinases necessary for generating the MCs is not possible in production strains of bacteria, as this would lead to premature elimination of the parental plasmids undergoing replication in the bacteria before they could be amplified in sufficient quantity to be available as a starting substrate for production of the MCs.

Since retaining the superhelical structure is essential to the subsequent use of the MCs, correspondingly stringent requirements apply to the purification method for isolating the MCs in order not to destroy the desired structure of the MCs during purification. In the conventional variant ((1), (3)), the MCs are separated from the DNA secondary products arising after recombination (parental plasmid, miniplasmid and concatemers of the individual products) by targeted linearisation of the unwanted molecules by means of specifically selected restriction enzymes and subsequent agarose gel electrophoresis. The MC is not modified by the restriction enzymes during this procedure, such that the circular status of the MCs is unaffected. There is, however, a significant risk of eluting unwanted DNA molecules, such as for example the (possibly linearised) parental plasmid, from the gel and purifying them together with the MCs (3). Since the parental plasmids, like the MCs, are capable of transducing eukaryotic cells, this may have a negative impact on the MC experiment (for example permanently transduced cells due to the integration of the parental plasmid into the host cell genome).

Other variants for purifying MCs generated by recombination in bacteria make use of a column chromatography system which exploits the binding of specific proteins to the lactose operator sequence (lacOs) which is present in this case in the MC (11, 12). In this case, the purity achieved and also the relative yield of the system are better than in the conventional variant of purification using agarose gel. In return, the drawback must be accepted that the MC then contains bacterial DNA sequences (lacOs), which undermines the functional (5, 6) and regulatory advantage of MCs over conventional plasmid vectors.

The method presented in the present invention for producing MCs in vitro is capable of overcoming the disadvantages of previously published MC production methods.

The novel method dispenses with the use of recombinases for generating the MCs from the parental plasmid. At the same time, it is not necessary to purify the resultant MCs by means of a complex and costly method such as for example column chromatography, since any mixing of the MCs with the various secondary products is avoided from the outset. This simplifies the production of MCs in comparison with the previously conventional methods while simultaneously increasing the yield and purity of the target molecules, in particular superfluous bacterial sequences being completely avoided in the backbone of the MC.

The method presented here is based on a combined in vivo/in vitro technology, in which circular superhelical minicircle DNA is generated in a final enzymatic step with the assistance of gyrase.

The present invention accordingly provides a method for producing a circular DNA vector in superhelical form, comprising the steps:
(a) cleaving a parental plasmid with one or more restriction enzymes, the parental plasmid containing the sequence of the DNA vector together with heterologous sequences, in order to obtain a linear DNA vector fragment with the sequence of the DNA vector,
(b) separating the linear DNA vector fragment from other restriction cleavage products,
(c) ligating the linear DNA vector fragment in order to obtain a circular DNA vector in relaxed form,
(d) separating the circular DNA vector from other ligation products,
(e) coiling the circular DNA vector from step (d) with a gyrase in order to obtain a circular DNA vector in superhelical form, and
(f) optionally purifying the circular DNA vector in superhelical form to separate it from secondary products.

The present invention furthermore provides a method for producing superhelical circular DNA vectors, comprising in vitro restriction cleavage, in vitro ligation and in vitro coiling with gyrase.

The present invention furthermore provides a method for producing superhelical minicircle DNA vectors without using site-specific (sequence-specific) recombinases such as for instance FLP.

In a preferred embodiment, the DNA vector, in particular the MC DNA vector, is produced as follows:
The necessary vector information is amplified together with heterologous sequences on the parental plasmid (PP) in a host cell, for example in a bacterium.
The parental plasmid is isolated from the host cell using standard methods.
After isolation, the MC sequences of the PP are excised from the PP by digestion with restriction enzymes. The resultant linear fragments, one of which contains the MC regions, are separated, for example by agarose gel electrophoresis. The linearised MC fragments isolated using conventional methods, for example by elution, and used for further MC production.
The isolated linearised MC DNA molecules are religated by using (recombinant) ligases, such that the MCs are obtained in the relaxed, “open circle” form.
Following ligation, the relaxed MCs are coiled using a (recombinant) gyrase in an in vitro batch, such that the corresponding superhelical (supercoiled) forms of the MCs are obtained, as are required for the use thereof for transducing eukaryotic cells.
Finally, by-products of the enzymatic gyrase reaction are removed from the circular, superhelical vector using standard methods (for example agarose gel electrophoresis with subsequent gel elution, Qiagen DNA purification kit).

The method described here for producing DNA vectors, in particular minicircle gene vectors, makes it possible to produce the DNA vectors, which may for example be used for producing recombinant cell lines or for gene therapy, in large quantities and with elevated purity.

The method according to the invention moreover yields novel preparations of MC vectors in elevated purity.

This results, on the one hand, in distinct cost and time savings for the previously described fields of use and moreover opens up prospects for novel fields of application.

A circular DNA vector is produced in superhelical form using the above-stated method. The DNA vector consists of double-stranded DNA and conventionally has a size of 0.5 to approx. 10 kB, in particular of approx. 1-6 kB. Smaller or larger DNA vectors may, however, also be produced. In a preferred embodiment, the DNA vector is a minicircle (MC) DNA vector, i.e. a circular DNA vector which contains the necessary functions for the DNA vector, for example the subsequent minicircle DNA vector. These functions conventionally contain sequences for a transgene, for example a recombinant gene or a corresponding cDNA, together with regulatory sequences for gene expression, for example transcription and translation initiation sequences such as promotors, enhancers, ribosomal binding sites, etc. and optionally transcription termination sequences such as polyA sequences and further regulatory or functional nucleotide sequences such as for example S/MAR sequences for transferring the adherence of the DNA vector to the nuclear matrix as an initiator for integration-independent episomal replication (2, 13, 14).

The DNA vector may contain a pharmaceutically usable DNA sequence as the transgene, for example a mammalian gene or cDNA, in particular a human gene or a recombinant variant thereof for therapeutic applications, or a synthetic sequence, for example a synthetic gene. A “transgene” or a “transgenic sequence”, these terms being usable interchangeably, which is preferred according to the invention is accordingly a eukaryotic sequence, in particular a human sequence and/or a synthetic sequence. Such a sequence for example encodes growth factors, cytokines, interleukins, interferons, tumour-suppressor proteins etc. On the other hand, the DNA vector may also contain a sequence from pathogenic organisms which encodes an antigen or a sequence which encodes a tumour antigen or autoantigen, as the transgene.

The preparations of circular superhelical DNA vectors, in particular MC vectors, obtainable by the method according to the invention may be used both as research reagents and as pharmaceutical products. Medical applications such as for instance DNA vaccination, gene therapy, cell reprogramming or RNAi insertion are preferred.

The DNA vectors, in particular MC vectors, produced by the method may furthermore also be used for producing therapeutic proteins in recombinant cells, in particular eukaryotic cells, in particular CHO cells. To this end, the corresponding host cells are transiently or stably transduced by the DNA vectors, such that the resultant recombinant cells produce the desired therapeutic protein. The therapeutic protein may here be produced using conventional methods of industrial biotechnology.

A further area of application of the DNA vectors, in particular minicircle vectors, produced using the method described herein is the genetic modification of host cells which are to be used for expressing recombinant proteins. The host cells are here modified by the DNA vectors in such a manner that expression of the transgenic protein in the modified host cells is superior in terms of quantity and/or quality to the expression of protein from unmodified host cells.

In addition, using DNA vectors, in particular MC vectors, enables subsequent modification of production cells which already produce recombinant protein, such that said cells may be desirably modulated by the genes introduced by the MCs with regard to transgene quantity and/or quality.

The corresponding target cells may be modified with the MCs produced by the described method by simultaneously transducing the target cells with one or more MCs.

The circular superhelical DNA vectors are generated from “parental plasmids”, which do not differ from usual plasmids with regard to their basic functions. They serve to amplify the DNA vector sequences in host cells, for example bacteria such as for instance E. coli. Other examples of suitable host cells are yeasts, such as for instance Saccharomyces. In addition to the DNA vector sequence, these parental plasmids conventionally contain heterologous sequences on a contiguous part of the complete plasmid, for example for propagation in a host cell, such as for instance information for the replication origin for initiating replication of the plasmid in host cells as well as, normally, a gene including regulatory sequences for transferring antibiotic resistance to host cells carrying the plasmid.

Since DNA vectors may be produced according to the present invention without DNA recombination mediated by site- or sequence-specific recombinases, a parental plasmid may be used as starting material which is free of recombinase recognition sequences, for example free of recognition sequences for sequence-specific recombinases, such as for instance FLP, Cre, RecA, Phi-C31 and others.

The parental plasmid (PP) therefore substantially consists of two parts:
The part with the functional units for replication and amplification of the parental plasmid in host cells, for example bacteria.
The second part with the information which is required for the DNA vector.

The plasmid and DNA vector sequences are separated by recognition sequences for one or more restriction enzymes which preferably do not occur on the DNA vector fragment.

The parental plasmid used as starting material for the method according to the invention is conventionally obtained by culturing a host cell, in particular a prokaryotic host cell, such as for instance E. coli, and isolating the plasmid from the host cell. The host cell used is preferably a strain of bacteria which is suitable for high-copy amplification of plasmids, such as for instance E. coli XL1 Blue (16). Parental plasmids are here recovered in a manner which does not differ from methods for producing conventional plasmids or DNA vectors.

The PPs from the bacteria may be purified using standard methods, for example using commercially obtainable kits (for example QIAgen Midiprep).

Step (a) of the method according to the invention involves cleaving a parental plasmid with one or more restriction enzymes. This step is favourably carried out in vitro, i.e. on an isolated plasmid preparation. The parental plasmid is cleaved using restriction enzymes which permit the excision of a fragment which includes the sequence of the DNA vector (DNA vector fragment). This DNA vector fragment is obtained in linear form in addition to one or more further linear fragments corresponding to the additional heterologous sequences present in the parental plasmid.

The region of the DNA vector is preferably not cut during restriction cleavage, while the remaining sequence, also known as miniplasmid (MP), is either left behind as a whole piece or is alternatively cleaved into smaller pieces. It is important here for no DNA fragments to be obtained which are similar in size to the DNA vector fragment. Restriction cleavage preferably proceeds quantitatively, such that no intact parental plasmid is any longer present after the enzymatic treatment of the DNA.

According to step (b), the linear DNA vector fragment is separated from other restriction cleavage products, i.e. other linear DNA fragments. Separation is conventionally performed by size, for example by gel electrophoresis. Separation by agarose gel electrophoresis is preferred. As the result of the separation, the linear DNA vector fragment is isolated in high purity form, free of other restriction cleavage products. Since the DNA vector fragment is present in linearised form, it may readily be identified on the basis of its size by means of a conventional DNA marker in the gel.

The linearised MC may be isolated from the agarose gel using standard methods, for example by elution and purification by means of commercial kits (for example QIAgen Gel Elute). The resultant DNA solely contains linearised MC DNA.

According to step (c), the linear DNA vector fragment is brought into contact with a ligase under conditions in which ligation proceeds, so giving rise to a circular DNA vector in relaxed form. Step (c) is favourably carried out in vitro, i.e. on an isolated preparation of the DNA vector fragment. Suitable ligases are commercially available, for example in recombinant form.

In order to ensure that ligation is as effective as possible, care should be taken during the previously carried out digestion of the PP that, where possible, the resultant 5′ and 3′ ends of the linearised DNA vector fragment have complementary nucleotide overhangs. Ligation may optionally also be carried out with smooth ends.

In order to prevent any linearised MP DNA, which may under certain circumstances have been entrained, from being circularised or a PP being reformed from linearised MC and linear MP, it is preferred when selecting the restriction enzymes for separating MC and MP to ensure that the MP is cut by means of an additional enzyme in such a manner that a complete MP fragment, which can be circularised with itself or with the MC, is not obtained.

After ligation, the batch is purified according to step (d) in order to separate the circular DNA vector from other ligation products. This may, for example, proceed by separation using an agarose gel. Any possible contamination with MP fragments or MC concatemers becomes visible at this point. Only the bands for the circularised DNA vector (MC) are excised from the gel and the DNA is eluted therefrom. In this way, the degree of purity of the MC DNA is further increased and contamination with MP or PP fragments is virtually ruled out.

If the MC is to be used as a DNA vector, specifically where it is to be used as a stably episomally replicating vector, it is necessary for the annular DNA molecule to assume superhelical form. This superhelical status is not obtained once circularisation with ligase is complete and must therefore be produced subsequently by gyrase treatment of the circular MC. The gyrase, which is commercially obtainable in recombinant form, is a type II topoisomerase which, in the presence of ATP, introduces negative superhelical structures into DNA.

Step (e) therefore involves contacting the circular DNA vector from step (d) with a gyrase under conditions in which coiling of the vector takes place, for example in the presence of ATP. In this manner, a circular DNA vector is obtained in superhelical form in elevated purity and yield. Step (e) is favourably carried out in vitro, i.e. on an isolated preparation of the DNA vector. The duration of the reaction may be varied in order to obtain preparations with a different degree of coiling.

The reaction may proceed in accordance with the manufacturer's instructions for use of the gyrase. Once the superhelical structures have been introduced into the MCs, the latter may finally be purified from the reaction batch by means of standard methods, for example by means of commercial kits (for example QIAgen MidiPrep).

Step (e) involves purifying the circular, superhelical DNA vectors by means of standard methods (precipitation, agarose gel electrophoresis with subsequent elution, Qiagen Qiaquick Nucleotide Removal Kit etc.) in order to separate secondary products from the enzymatic gyrase reaction.

Once the MCs have been produced using the method described here, there are various options for testing the success and quality of the MC preparation:
Agarose gel electrophoresis. Only one band should be visible here, the size of which may be determined with the assistance of a DNA marker for superhelical DNA.
Verification of the superhelical structure, for example by means of a chloroquine gel. Using a gel of this kind, circular DNA may be separated into its various forms as a function of the number of introduced superhelical structures (10).
PCR may be used to verify with very high sensitivity whether exclusively circular MC structures are present in the preparation, or whether there is also contamination with linear or circular MP and/or PP.

The present invention also provides a reagent kit which contains restriction enzymes, a ligase and a gyrase for carrying out the method according to the invention. The kit according to the invention kit preferably comprises a restriction endonuclease. In a further, preferred embodiment no endonuclease is present. A restriction endonuclease is particularly preferably included while an exonuclease is simultaneously absent. In a preferred form of the invention, the kit comprises a set of instructions for carrying out the method according to the invention.

The present invention furthermore provides a preparation of a DNA vector, in particular of a minicircle DNA vector in superhelical form, characterised by the absence of secondary products, in particular linear or circular miniplasmid and/or parental plasmid. According to PCR, the preparation preferably contains no sign of the above-stated secondary products. The PCR reaction is here not part of the MC production process, but instead a detection method for contamination by parental plasmids and miniplasmids, which makes it possible to demonstrate the greater purity of the inventive MC preparations which are produced in vitro in comparison with MC preparations produced in the conventional manner by recombination.

In order to detect contaminant parental plasmids by means of PCR, the sequence-specific primers must be selected such that one of the two oligonucleotides binds in the region of the heterologous backbone of the parental plasmid, while the corresponding primer is located in the region of the minicircle. With this arrangement of the oligonucleotides, the corresponding fragment is only amplified if parental plasmid is present in the MC preparation. Contaminant miniplasmids may also be found with the assistance of a second PCR. To this end, both PCR primers must be located in the region of the heterologous backbone of the original parental plasmid. If the PCR-specific fragment is amplified, it may originate either from the miniplasmid or from the parental plasmid. If the first PCR was negative for parental plasmid, the amplification product of the second PCR should be attributed to miniplasmid. If both PCR reactions are positive, it is not possible to make an unambiguous statement with regard to the nature of the secondary products.

The minicircles produced in vitro have a content of superhelical structures which is purposefully controllable. The consequent predictability of the composition of the in vitro MC preparation is distinctly superior to the random composition of circular DNA molecules obtained from bacteria by site-specific recombination. This superiority has substantial consequences, in particular for the use of therapeutic vectors in clinical applications.

The present invention will now be illustrated by the following figures and examples.

### KEY TO THE FIGURES

FIG. 1: Plasmid Map of the MC Parental Plasmid “pEpi-eGFP M18 antiHLC” and of the Recombination Products.

The parental plasmid contains all the units necessary for a functional plasmid, such as origin of replication (ori, in this case between HSV TK polyA and the following FRT site; not shown in the figure) and antibiotic resistance (Neo/Kana). A vital factor when producing MC vectors by the conventional method by means of sequence-specific recombination is the presence of both FRT sequences which serve as points of attack for the FLP recombinase. FLP-induced recombination of these two sequences with one another results in two independent circular DNA molecules being pinched off. One of these carries the information for the functions of importance in bacteria and is therefore designated as miniplasmid. The second molecule no longer contains any functional bacterial sequences, but instead only carries vector-specific information and is thus the minicircle.

FIG. 2: Comparison of the “In Vitro” Minicircle with the Minicircle Produced by Site-Specific Recombination in E. coli EL250 with the Assistance of a 1% Agarose Gel.

The in vitro MC was obtained from the PP by restriction with the enzyme XbaI and subsequent religation with a T4 ligase. The ligated DNA (in each case 1 μg) was then treated for 30 min (track 1), 2 h (track 2) or 5 h (track 4) with 1 U of DNA gyrase.

The “EL250” MC was obtained by site-specific recombination in E. coli EL250 after induction with 0.3% L-arabinose (track 3). The in vitro MC and the “EL250” MC have the same running behaviour in a 1% agarose gel, i.e. superhelical structures were introduced into the ligated MC DNA with the assistance of the DNA gyrase.

It can be seen in tracks 1, 2 and 4 that the ligated MC had been completely converted into the supercoiled (ccc) form after just 30 minutes' gyrase treatment. MC concatemers and non-ligated MC DNA can also be seen. A strong PP band and an MP band may be seen in track 3 in addition to the MC band.

Key: i.v.=in vitro minicircle; EL250=minicircle produced by site-specific recombination in E. coli EL250; MC=minicircle; PP=parental plasmid; MP=miniplasmid

FIG. 3: Separation of Various ccc Plasmids on a 0.8% Chloroquine/Agarose Gel

Plasmids pMAXGFP (LONZA), CMV-GFP parental plasmid and pEpi-delCM18opt (Rentschler Biotechnologie) were used. The agarose gel contained 2.5 μg mL⁻¹ chloroquine. The gel was run for 15 h at 2.5 V cm⁻¹. (A) Either 1 μg or 500 ng of each plasmid was used for the gel run. A characteristic band pattern was obtained for all the plasmids used. (B) The signals were quantified using the ImageJ software package.

FIG. 4: Separation of Various Quantities of the ccc Plasmid pEpi-delCM18Opt on a 0.8% Chloroquine/Agarose Gel.

The DNA was separated for 15 h at 2.5 V cm⁻¹. The gel contained 2.5 μg mL⁻¹ chloroquine. (A) Rows 1-6 contain various quantities of ccc DNA, while row 7 contains the linearised plasmid. (B) The band patterns were recorded using the ImageJ software package. It is clear that applying a quantity of 100 ng DNA is sufficient for evaluation of the band patterns. Given a longer exposure, even as little as 50 ng may be evaluated (data not shown).

FIG. 5: Comparison of Coiling of the “In Vitro” Minicircle with the Minicircle Obtained by Site-Specific Recombination from Plasmid pEpi-delCM18Opt

(A) Track 1: The in vitro minicircle was produced by restriction digestion of the parental plasmid with the enzymes XbaI and BstBI and subsequent ligation (T4 ligase) (oc). It may be seen that ligation is not complete, a certain proportion of the DNA remaining linearised. Concatemers of two or more DNA fragments are also obtained. Track 2: The gyrase and the gyrase buffer were added directly to the ligation batch (1 μg DNA). It may be seen that no coiling has taken place. Track 3: The ligation was firstly purified (QIAGEN PCR Purification Kit), then 1 μg of ligated DNA was treated for 2 h with 5 U of gyrase. Track 4: The minicircle was produced by site-specific recombination. Recombination was induced by L-arabinose in E. coli EL250 with the assistance of an FLP recombinase encoded in the genome.

(B) Tracks 3 and 4 from (A) were shown in enlarged, inverted form here. The MC obtained by site-specific recombination (track 4) shows the expected band pattern. The in vitro MC (track 3) likewise shows the band pattern, but one band is particularly pronounced.

(C) The signals were recorded using the ImageJ software package and are indicated correspondingly with arrows. The distinct band signal mentioned in (B) can be seen in track 3 (red arrow). Key: linear=linearised DNA, oc=open circle DNA, ccc=supercoiled DNA, U=unit, EL250=MC produced by site-specific recombination, MC=minicircle

FIG. 6: Verification of the Purity of the Generated Minicircles by PCR and 1% Agarose Gel

In order to check the generated minicircle DNA from pEpi-delCM18opt (vector map A) for possible contamination with parental plasmid or miniplasmid, a fragment in the miniplasmid region (see linearised vector map B; PCR 1) and over an FRT site (see linearised vector map B, PCR 2) was amplified by PCR. 10 ng of template DNA were used in each case. If an amplification product is obtained in PCR 1, the sample contains either miniplasmid or parental plasmid, while if an amplification product is obtained in PCR 2, the sample contains parental plasmid, since in this PCR there is one primer located in the minicircle region and one primer in the miniplasmid region. (C1) An amplification product was obtained in the sample with the minicircle produced by site-specific recombination (EL250 MC), while no amplification product was obtained in the sample with the in vitro minicircle. (C2) An amplification product was likewise obtained in the case of the EL250 minicircle. Key: PP=parental plasmid; MC=minicircle, EL250 MC=minicircle produced by site-specific recombination in E. coli strain EL250.

FIG. 7: Sequencing of the “In Vitro” Minicircle from pEpi-delCM18Opt

(A) Vector map of the pEpi-delCM18opt MC with sequenced region (red arrow) (B) Comparison of the sequence of the in vitro MC with the sequence according to the vector map using the ClustalW software package (EMBL-EBI). Key: orange=SV40 promotor/enhancer; brown=FRT site; blue=XbaI restriction site within the FRT site; green=eGFP gene; *=base match.

### EXAMPLE

Production and characterisation of MC using an in vitro method with ligase and gyrase

Using ligase and gyrase, superhelical MCs could be produced successfully in vitro without having to depend on the use of sequence-specific recombinases or specific strains of bacteria for replicating the PP and subsequent induction of MC production.

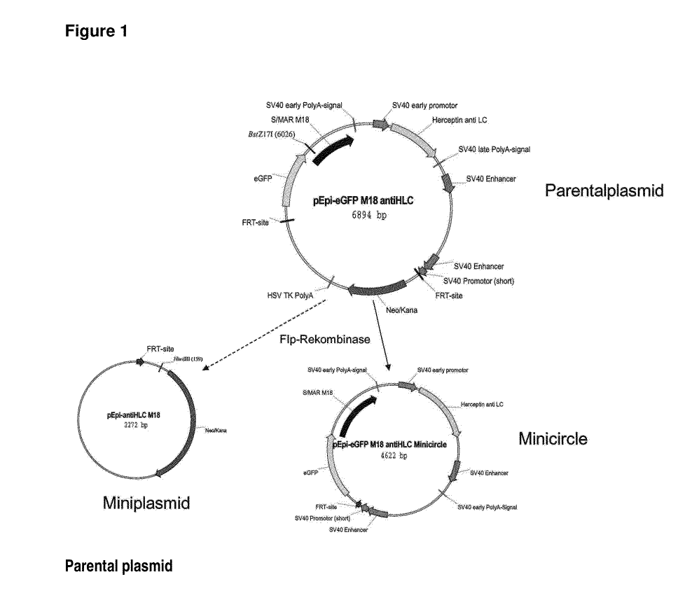

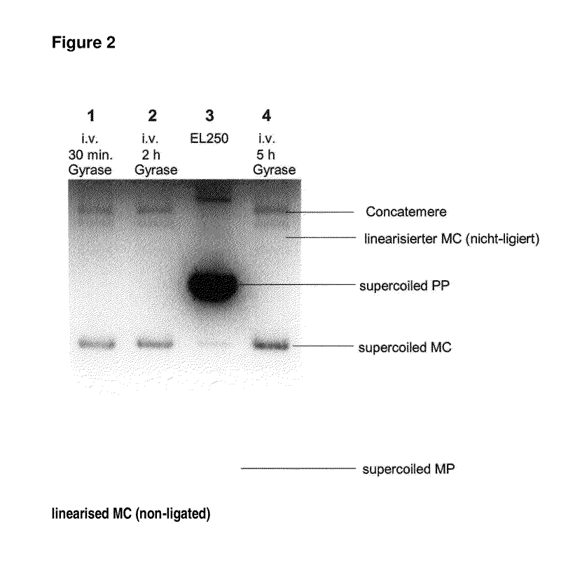

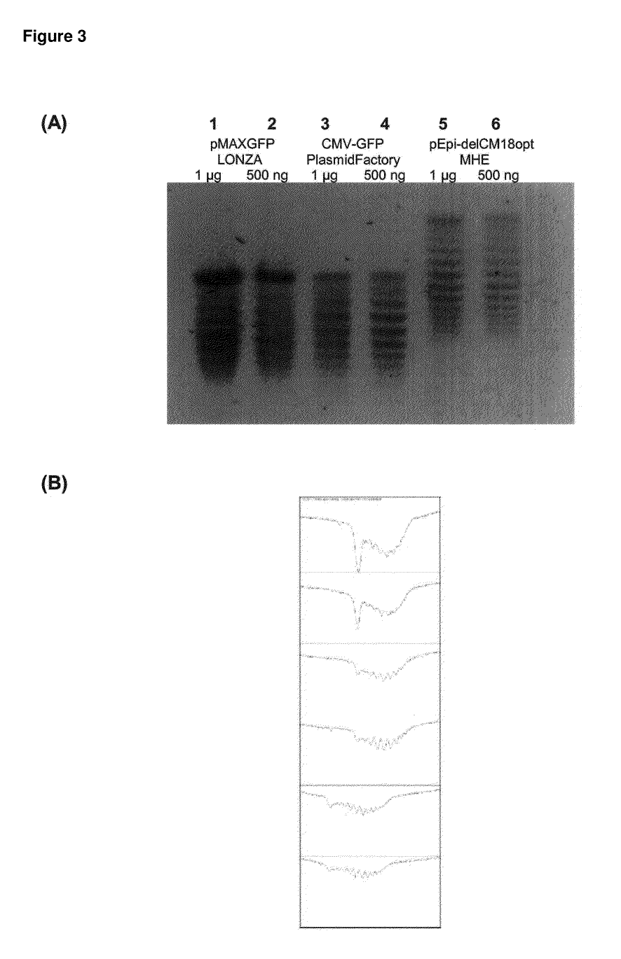

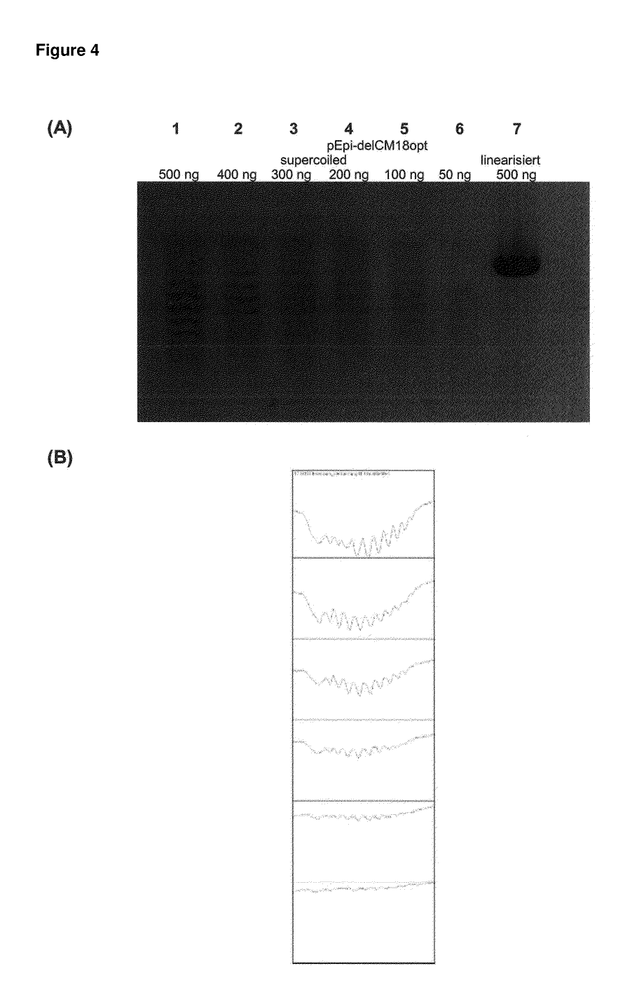

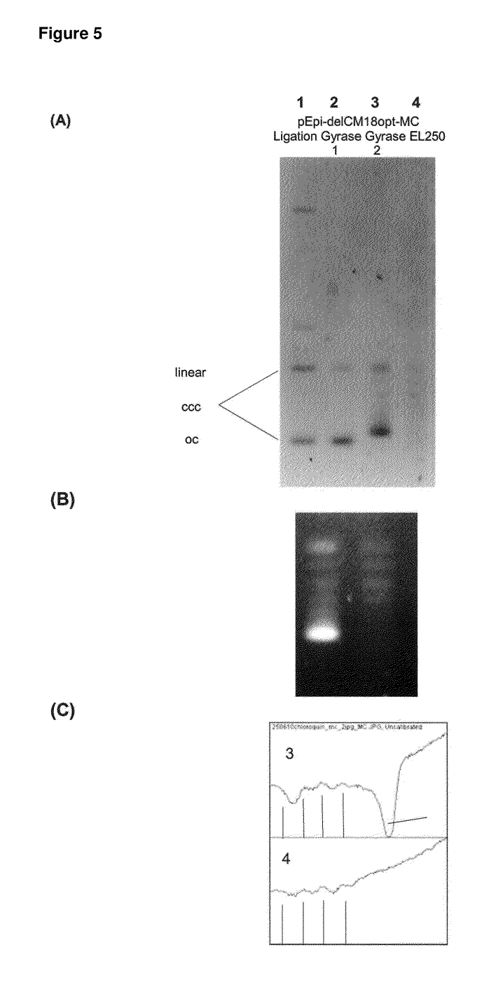

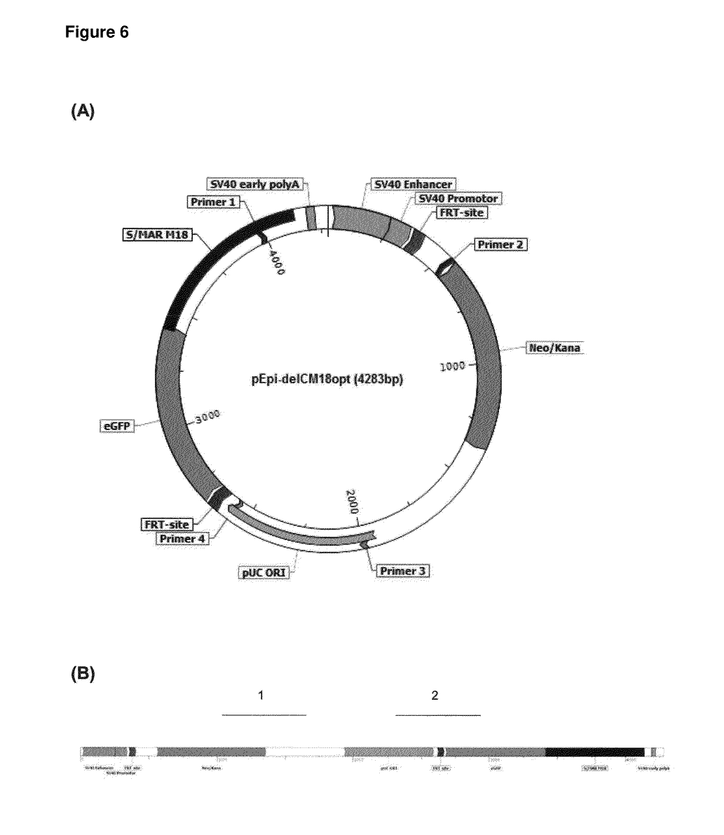

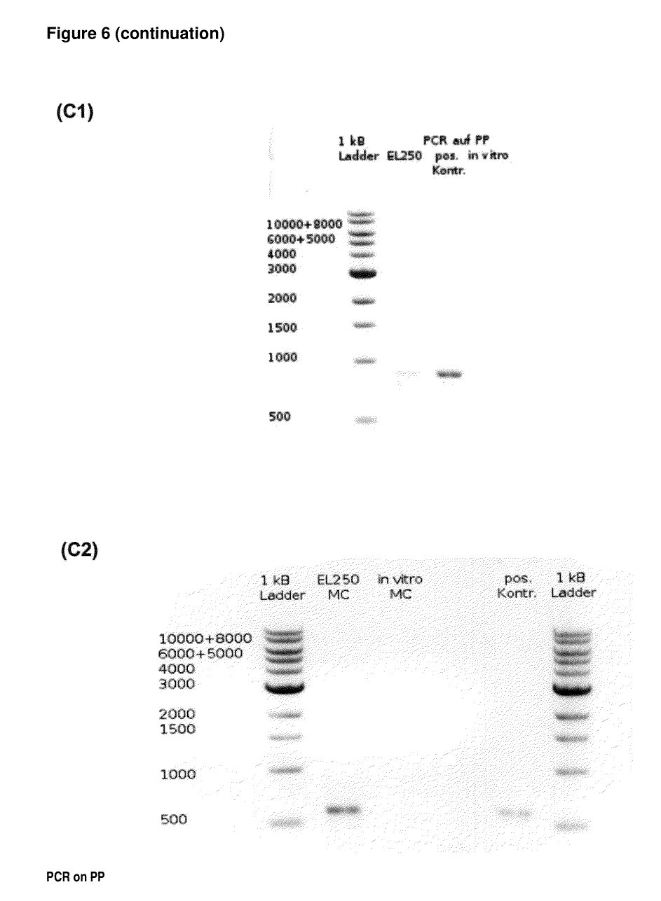

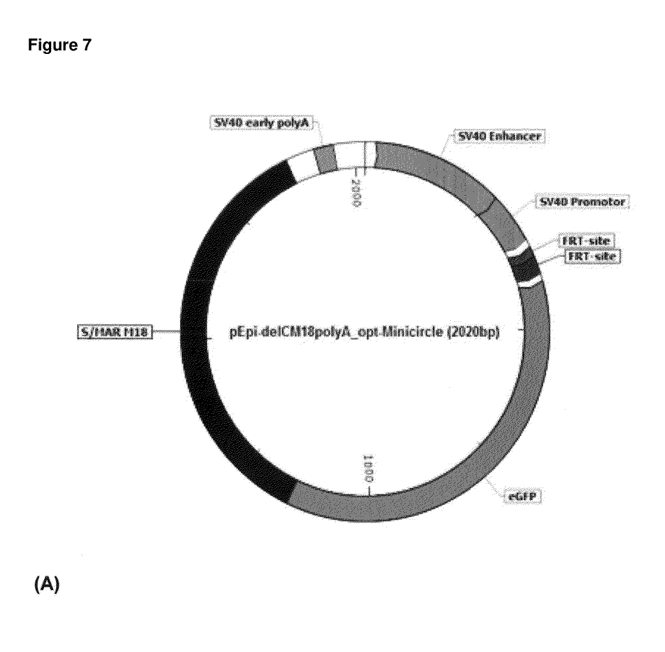

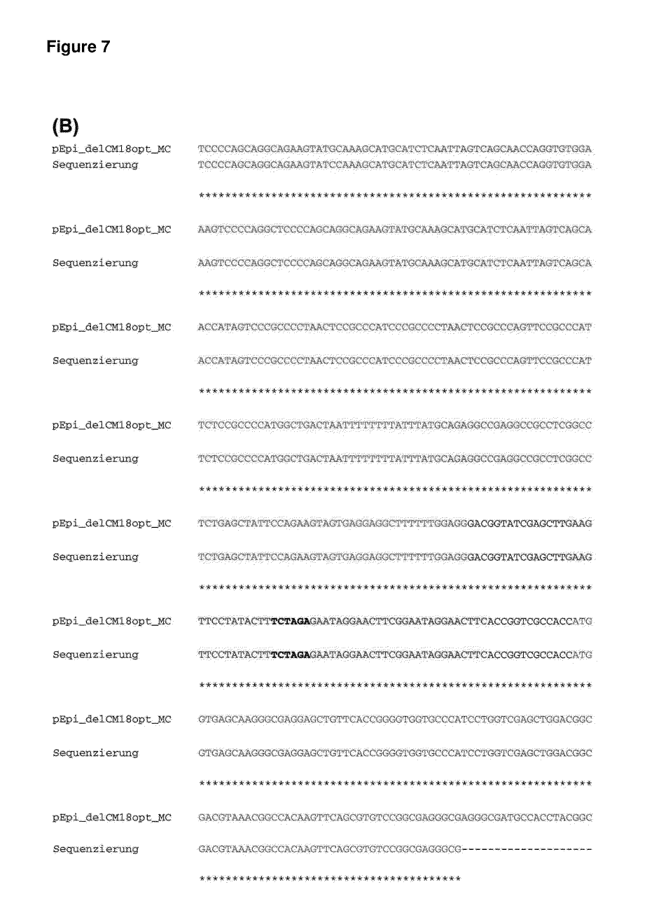

A PP (pEpi-eGFM18 anti HLC) was used for testing purposes which has all the elements which are required for conversion into MC and MP with the assistance of induced, sequence-specific recombination. This enabled a direct comparison of the two methods. In principle, given suitable restriction digestion, any plasmids may be converted into MCs with the in vitro method. FIG. 1 shows a schematic diagram of this plasmid.

1. Production of MC from 50 mL of Bacterial Culture: Comparison of Method According to the Invention (In Vitro) and Recombinase-Mediated Method

The semi-synthetic minicircle DNA vectors described in this application were produced using the method described in greater detail below: the parental plasmid, in the case shown here pEpi-delCM18opt, consisting of miniplasmid and minicircle region, was introduced by electrotransformation into E. coli XL1 Blue(¹⁶). After selecting individual clones on agar plates (using an appropriate selective medium), the respective plasmid DNA of the clones was investigated for the correct base sequence by restriction digestion and sequencing. Long-term storage of the correct clones was achieved by mixing a 5 mL overnight culture with 87% glycerol in a 1:1 ratio and storing at −20° C. (glycerol stock).

The parental plasmid was generated for subsequent in vitro production of minicircle DNA by transferring some of the culture from the glycerol stock into an Erlenmeyer flask containing LB selective medium and cultured overnight at 37° C. and 180 rpm on an orbital shaker. The plasmid DNA (=parental plasmid) was recovered by centrifuging the culture at 6000×g for 15 min. The plasmid DNA was prepared using a QIAGEN Plasmid Kit in accordance with the manufacturer's instructions.

The minicircle, which is flanked by two identical restriction sites, was excised from the plasmid DNA by overnight restriction digestion. The products of restriction digestion were separated by gel electrophoresis (1% agarose gel). After the gel run, the DNA in the gel was stained with methylene blue and the gel fragment with the linearised minicircle DNA was excised with a scalpel. The minicircle was recovered from the gel using the QIAGEN Gel Extraction Kit in accordance with the manufacturer's instructions.

The linearised minicircle DNA was religated at 16° C. for 16 h by a T4 ligase. The ligated DNA from the ligation batch was purified by agarose gel electrophoresis using the QIAGEN Gel Extraction Kit in accordance with the manufacturer's instructions.

Conversion of the ligated minicircle into the ccc state was achieved by incubation with a DNA gyrase at 37° C. The incubation time was here determined on the basis of the desired degree of coiling and was between 30 min and 24 h. The batch was separated by gel electrophoresis (1% agarose). The DNA was here stained with the assistance of methylene blue and the gel fragment comprising the ccc minicircle DNA was excided from the gel with a scalpel. The minicircle was recovered from the gel using the QIAGEN Gel Extraction Kit in accordance with the manufacturer's instructions.

Production of the recombination-induced minicircles from the same parental plasmid (pEpi-delCM18opt) as the semi-synthetic minicircles was carried out as follows by the conventional method using recombinases:

FlpE recombinase-induced recombination proceeds between two “FRT” sites, which in this case flank the minicircle sequence located in the parental plasmid. The E. coli strain EL250(²) contains the gene for FlpE recombinase integrated in its genome. This gene is under the control of an L-arabinose-inducible promotor, i.e. expression of this gene is only switched on once L-arabinose has been added to the culture medium. Induction proceeds in M9 minimal medium, since glucose or sucrose in the LB medium would disrupt L-arabinose uptake.

The parental plasmid, consisting of miniplasmid and minicircle region, was introduced into E. coli EL250 by electrotransformation. After selecting individual clones on agar plates (using an appropriate selective medium), the respective plasmid DNA of the clones was investigated for the correct base sequence by restriction digestion and sequencing. Long-term storage of the correct clones was achieved by mixing a 5 mL overnight culture with 87% glycerol in a 1:1 ratio and storing at −20° C. (glycerol stock).

Prior to L-arabinose induction, some culture from the glycerol stock was transferred into an Erlenmeyer flask with LB selective medium and cultured at 30° C. for 24 h. The culture was then centrifuged at 3700×g for 15 min. The pellet was resuspended or washed in M9 minimal medium (½ of the previous volume) and centrifuged again for 15 min at 3700×g. The pellet was then resuspended in the original culture volume in M9 minimal medium with addition of 0.3% L-arabinose and incubated in an Erlenmeyer flask for 5 h at 30° C. and 180 rpm on an orbital shaker. In this way, Flp recombinase expression was induced, which in turn catalysed recombination of the parental plasmid into minicircles and miniplasmids (this reaction does not proceed quantitatively in the bacteria, such that residues of unreacted parental plasmid always remain in the bacteria).

The non-chromosomal DNA (=minicircle, parental plasmid and miniplasmid) was recovered by centrifuging the culture at 6000×g for 15 min. The plasmid DNA was prepared using a QIAGEN Plasmid Kit in accordance with the manufacturer's instructions.

The unreacted parental plasmid and the miniplasmid obtained as a by-product which was not required were linearised by overnight restriction digestion using a suitable restriction enzyme which only cuts in the miniplasmid region. The minicircle was left unchanged by this reaction and therefore remained in the ccc state. The products of restriction digestion were separated by gel electrophoresis (1% agarose gel+0.5 pg/ml ethidium bromide). The gel fragment comprising the ccc minicircle DNA was excised with a scalpel on a UV table. The minicircle was recovered from the gel using the QIAGEN Gel Extraction Kit in accordance with the manufacturer's instructions.

The initial volume of the bacterial cultures was 50 mL in both cases.

As is clearly visible in FIG. 2, the yield of MC DNA relative to the initial volume of the bacterial cultures is approx. 10-15 times higher in the method according to the invention than in the method involving sequence-specific recombination. The reasons for this are in particular the higher initial number of PP copies per bacterium in the strain of bacteria used for the method according to the invention and the lower efficiency of sequence-specific recombination in comparison with ligation (clearly visible from the strong PP band in the gel).

2. Detection of the Superhelical Status of In Vitro Produced Minicircle

The superhelical status of a DNA molecule indicates the extent to which the double helix is itself further coiled. This “coiling status” is regarded as decisive for the efficiency of DNA vectors with regard to their capability for transforming eukaryotic cells with a significant impact on stability of expression and integration into the host cell genome.

2.1 Superhelical Status of Plasmid DNA Isolated from Bacteria

An agarose gel to which a specific concentration of chloroquine has been added is suitable for investigating the degree of coiling of ccc DNA (10). Chloroquine inserts positive “supercoils” into the negative ccc DNA. Moreover, the ccc DNA band is separated as a function of the tightness of coiling. The method was first established. To this end, the ccc plasmids pMAXGFP (LONZA, Nucleofector Kit), CMV-GFP parental plasmid (Plasmid Factory) and pEpi-delCM18opt (Rentschler Biotechnologie) were separated on a 0.8% agarose gel with 2.5 μg m1⁻¹ of chloroquine for 15 h at 2.5 V cm⁻¹ (FIG. 3).

Both the commercial plasmid samples and the self-prepared plasmid DNA from E. coli XL1 Blue (pEpi-delCM18opt) are a mixture of a plasmid with various “degrees of coiling”. It is clear from the graph that there is no uniform coiling index which predominates in prokaryotes, each plasmid having its own band pattern. At a chloroquine concentration of 2.5 μg ml⁻¹, ccc DNA with an elevated coiling index runs faster than one with a low coiling index. The DNA behaviour is reversed at higher chloroquine concentrations.

2.2 Comparison of the Superhelical Status of MC from Site-Specific Recombination and of (In Vitro) MC According to the Invention

In order to permit a comparison of the coiling of the minicircles obtained by site-specific recombination with the in vitro minicircles, the smallest quantity of DNA to be used was determined, since the yield of minicircles by site-specific recombination is very low. 50-500 ng dilutions of the ccc parental plasmid pEpi-delCM18opt were used for this purpose (FIG. 4).

50 ng of DNA are sufficient to separate the ccc DNA in order to be able to evaluate the resultant band pattern with ImageJ. The band patterns of the MCs produced by site-specific recombination in E. coli and of the in vitro MCs were compared in order to demonstrate the suitability of the gyrase in the in vitro generation of ccc DNA for achieving the various coiling indices.

The degree of coiling of ccc DNA was investigated by separating the ccc DNA with the assistance of a chloroquine/agarose gel.

In order to generate the in vitro MCs, an investigation was carried out to determine whether the ligase may be used together with the gyrase in a reaction batch. As may be inferred from FIG. 5 (A, track 2), this is not possible under the tested reaction conditions. Gyrase treatment had no effect in this case. Purification of the ligation with subsequent gyrase treatment, on the other hand, provided a positive result, i.e. ccc DNA (FIG. 5, A, track 3). A comparison of the two MCs reveals that both MCs have a comparable band pattern, but the band is particularly pronounced for the in vitro MC. A further advantage of the novel method is that the quality of coiling of the in vitro MCs may be directly adjusted by the quantity of gyrase used and the incubation time. In the present case, a large quantity of gyrase (5 U) was used and a long incubation time selected, in order to obtain a uniform band pattern which reflects a uniform distribution of different degrees of coiling (details stated by the gyrase manufacturer New England Biolabs: 1 U of gyrase coils 0.5 μg of DNA in 30 min).

3. Verification of the Purity of the In Vitro MC Preparations by PCR

Differently produced MC preparations were compared in order to demonstrate the higher purity of our novel method relative to the various other methods. To this end, a PCR was in each case carried out on the miniplasmid region and over one of the two FRT recognition sites present in the PP in order to detect any parental plasmid which may possibly be present (FIG. 6). MCs which had been generated from the PP pEpi-delCM18opt were used for this purpose.

The PCR reactions for detecting miniplasmid and/or parental plasmid contamination in the minicircle preparation were, in the case presented in the present application, carried out as described below:

PCR 1—Detection of miniplasmid or parental plasmid: 10 pmol of “primer 3” (TTTTCTGCGCGTAATCTGCT) and “primer 4” (GTAAAAAGGCCGCGTTGCT) were used in each reaction. These were used, in order to amplify any contamination, with 10 ng of minicircle preparation or parental plasmid control DNA using RedTaq polymerase (Invitrogen) by means of following programme:

| Temperature | Time | | |
|---|---|---|---|
| [° C.] | [s] | | |
| 95 | 120 | | |
| 95 | 60 | | |
| 50 | 60 | {close oversize brace} | 30 cycles |
| 72 | 60 | | |

| | |
|---|---|
| 72 | 600 |
| 4 | 300 |
| 16 | ∞ |

The parental plasmid pEpi-delCM18opt corresponding to the minicircles was used as positive control. The amplification product to be expected in the event of contamination of the preparation has a size of 602 bp.

PCR 2—Detection of parental plasmid: 10 pmol of “primer 1” (GCATGCCATCATGACTTCAG) and “primer 2” (CGAAACGATCCTCATCCTGT) were used in each reaction. These were used, in order to amplify any contamination, with 10 ng of minicircle preparation or parental plasmid control DNA using RedTaq polymerase (Invitrogen) by means of following programme:

| Temperature | Time | | |
|---|---|---|---|
| [° C.] | [s] | | |
| 95 | 120 | | |
| 95 | 60 | | |
| 56.5 | 60 | {close oversize brace} | 30 cycles |
| 72 | 90 | | |

| | |
|---|---|
| 72 | 600 |
| 4 | 300 |
| 16 | ∞ |

The parental plasmid pEpi-delCM18opt corresponding to the minicircles was used as positive control. The amplification product to be expected in the event of contamination of the preparation has a size of 876 bp.

Surprisingly, only the minicircle produced using the conventional method in E. coli EL250 contains contamination with parental plasmid and possibly also miniplasmid. The in vitro minicircle produced using the novel method no longer contains any MP or PP DNA contamination.

4. Checking of Correct Assembly of the In Vitro MC by the Ligase

The in vitro MC was partially sequenced to investigate whether the MC also corresponds with regard to its sequence to the MC generated by site-specific recombination. To this end, the region of the FRT site obtained after recombination was sequenced (FIG. 7), MC and MP firstly being separated by the restriction enzyme XbaI, after which the MC fragment was religated by circularisation to form the desired in vitro MC.

Data from the partial in vitro MC sequencing revealed that the FRT region of the in vitro MCs corresponds to that of the FRT region of the vector map. Restriction with XbaI followed by ligation gives rise, as in site-specific recombination, to a new FRT site. This thus impressively demonstrates the superiority of the novel method. As expected, religation of the linear MC fragments to form circular in vitro MCs functions reliably.

5. Summary of the Present Results

The data in the present application show that it is possible, without using site-specific recombinases, to produce MC DNA vectors in vitro, the structural properties of which correspond to those of MCs produced conventionally by site-specific recombination.

The MCs produced by the in vitro method are distinguished by significantly higher purity while the production method is distinguished by a distinctly higher yield relative to the initial volume of the bacterial cultures.

By dispensing with site-specific recombination for production of the MCs, any plasmid DNA vectors may in principle be used as a starting material for producing MCs. The in vitro method does not involve cloning of the desired vector sequences (“gene of interest”) into specific parental plasmids with corresponding recombination sequences.

### Bibliography

(1) Bigger Brian W. et al. 2006, Methods of making minicircles, United States Patent Application 20060211117
(2) Broll Sandra 2009, Selbstreplizierende nicht-virale Episomen (Minicircles): Expressionseigenschaften and Etablierung im Zellkern [self-replicating non-viral episomes (minicircles): expression characteristics and establishment in cell nucleus], dissertation
(3) Broll Sandra et al. 2010, Minicircle Performance Depending on S/MAR-Nuclear Matrix Interactions, Journal of Molecular Biology, Volume 395, Issue 5, 950-965
(4) Che-Kun James C. et al. 1986, DNA supercoiling of recombinant plasmids in mammalian cells, Proc. Natl. Acad. Sci. USA, Vol. 83, pp 1641-1645
(5) Chen Zhi-ying et al. 2003, Minicircle DNA Vectors Devoid of Bacterial DNA Result in Persistent and High Level Transgene expression in vivo; Molecular Therapy, Vol. 8, 495-497
(6) Chen Zhi-ying et al. 2004, Silencing of episomal transgene expression by plasmid bacterial DNA elements in vivo, Gene Therapy, Vol. 11, 856-864
(7) Chen Zhi-ying et al. 2005, Improved Production and

Purification of Minicircle DNA Vector Free of Plasmid Bacterial Sequences and Capable of Persistent Transgene Expression In Vivo; Human Gene Therapy, Vol. 16, 126-131
(8) Chen Zhi-ying et al. 2010, Minicircle DNA vector preparations and methods of making and using the same, United States Patent Application 20100075401
(9) Esposito Franca et al. 1987, Supercoiling in prokaryotic and eukaryotic DNA: changes in response to topological perturbation of plasmids in E. coli and SV40 in vitro, in nuclei and in CV-1 cells, Nucleic Acids Research, Vol. 15 Num. 13, 5105-5124, 1987
(10) Goldstein et al. 1984, Regulation of bacterial DNA supercoiling: plasmid linking numbers vary with growth temperature, Proc. Natl. Acad. Sci. USA, Vol. 81, 4046-4050
(11) Mayrhofer Peter et al. 2007, Minicircle vector production, United States Patent Application 20070031378
(12) Mayrhofer Peter et al. 2008, Minicircle-DNA production by site specific recombination and protein-DNA interaction chromatography; The Journal Of Gene Medicine, Vol. 10, 1253-1269
(13) Nehlsen Kristina 2004, Molekulare Grundlagen der episomalen Replikation: Charakterisierung zirkulärer, nichtviraler Vektoren [molecular bases of episomal replication: characterisation of circular, non-viral vectors]; dissertation
(14) Nehlsen Kristina et al. 2006, Replicating minicircles: Generation of nonviral episomes for the efficient modification of dividing cells, Gene Ther Mol Biol Vol 10, 233-244
(15) Zechiedrich L. et al. 2009, Generation of minicircle DNA with physiological supercoiling, U.S. Pat. No. 7,622,252
(16) Bullock W O, Fernandez J M, J M Short. 1987. XL1-blue. A high efficiency plasmid transforming recA Escherichia coli strain with beta-galactosidase selection. Biotechniques 5: 376-378

1. A method for producing a circular DNA vector in superhelical form, comprising the steps:
(a) cleaving a parental plasmid with one or more restriction enzymes, the parental plasmid containing the sequence of the DNA vector together with heterologous sequences, in order to obtain a linear DNA vector fragment with the sequence of the DNA vector,
(b) separating the linear DNA vector fragment from other restriction cleavage products,
(c) ligating the linear DNA vector fragment in order to obtain a circular DNA vector in relaxed form,
(d) separating the circular DNA vector from other ligation products,
(e) coiling the circular DNA vector from step (d) with a gyrase in order to obtain a circular DNA vector in superhelical form, and
(f) optionally purifying the circular DNA vector in superhelical form to separate it from secondary products.

2. A method according to claim 1, characterised in that the parental plasmid is obtained by culturing a host cell, in particular a prokaryotic host cell, and isolating the plasmid from the host cell.

3. A method according to claim 2, characterised in that the parental plasmid is obtained from the host cell in circular form.

4. A method according to claim 1, characterised in that a parental plasmid is used which is free of recombinase recognition sequences.

5. A method according to claim 1, characterised in that the heterologous sequences of the parental plasmid comprise the sequences necessary for propagation in a host cell, in particular a prokaryotic host cell.

6. A method according to claim 1, characterised in that a DNA vector is produced which is free of prokaryotic sequences.

7. A method according to claim 1, characterised in that the DNA vector contains a transgene operatively linked with regulatory sequences.

8. A method according to claim 1, characterised in that a DNA vector is produced which contains S/MAR sequences.

9. A method according to claim 1, characterised in that the restriction cleavage in step (a) is carried out in vitro.

10. A method according to claim 1, characterised in that step (b) comprises a gel electrophoresis, in particular an agarose gel electrophoresis.

11. A method according to claim 1, characterised in that the ligation in step (c) is carried out in vitro.

12. A method according to claim 1, characterised in that the coiling in step (e) is carried out in vitro.

13. A reagent kit for producing a circular DNA vector in superhelical form, in particular for use in a method according to claim 1, comprising
(a) a ligase,
(b) a gyrase, and
(c) optionally one or more restriction enzymes.

14. A preparation of a minicircle DNA vector in superhelical form, characterised by the absence of linear or circular miniplasmid and/or parental plasmid.

15. The preparation according to claim 14, wherein the minicircle DNA vector is produced by the steps of:
(a) cleaving a parental plasmid with one or more restriction enzymes, the parental plasmid containing the sequence of the DNA vector together with heterologous sequences, in order to obtain a linear DNA vector fragment with the sequence of the DNA vector,
(b) separating the linear DNA vector fragment from other restriction cleavage products,
(c) ligating the linear DNA vector fragment in order to obtain a circular DNA vector in relaxed form,
(d) separating the circular DNA vector from other ligation products,
(e) coiling the circular DNA vector from step (d) with a gyrase in order to obtain a circular DNA vector in superhelical form, and
(f) optionally purifying the circular DNA vector in superhelical form to separate it from secondary products.

16. A method for producing superhelical circular DNA vectors, comprising (a) restriction cleavage of a parental plasmid in vitro, in order to obtain a linear DNA vector fragment with the sequence of the DNA vector, (b) ligation of the linear DNA vector fragment in vitro, in order to obtain a circular DNA vector in relaxed form, and (c) coiling of the circular DNA vector in relaxed form with gyrase in vitro.

17. A method according to claim 1, wherein the circular DNA vector is a minicircle DNA vector.

18. A method according to claim 1, characterised in that the method is carried out without using site- and/or sequence-specific recombinases, such as for example FLP.


SEQUENCE LISTING (6 sequences)

SEQ ID NO: 1 - DNA, 20 bp, Artificial Sequence
  Primer 3
ttttctgcgcgtaatctgct

SEQ ID NO: 2 - DNA, 19 bp, Artificial Sequence
  Primer 4
gtaaaaaggccgcgttgct

SEQ ID NO: 3 - DNA, 20 bp, Artificial Sequence
  Primer 1
gcatgccatcatgacttcag

SEQ ID NO: 4 - DNA, 20 bp, Artificial Sequence
  Primer 2
cgaaacgatcctcatcctgt

SEQ ID NO: 5 - DNA, 480 bp, Artificial Sequence
  pEpi_delCM18opt_MC
tccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggacggtatcgagcttgaagttcctatactttctagagaataggaacttcggaataggaacttcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggc

SEQ ID NO: 6 - DNA, 460 bp, Artificial Sequence
  Sequence of the in vitro minicircle according to vector map
tccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggacggtatcgagcttgaagttcctatactttctagagaataggaacttcggaataggaacttcaccggtcgccaccatggtgagcaagggcgaggagctgttcaccggggtggtgcccatcctggtcgagctggacggcgacgtaaacggccacaagttcagcgtgtccggcgagggcg